(19) United States
(12) Patent Application Publication
(10) Pub. No.: US 2016/0374591 A1
(43) Pub. Date: Dec. 29, 2016

(54) BANDSTRUCTURE CASCADE LASER CAPNOGRAPHY

(71) Applicant: ORIDION MEDICAL 1987 LTD., Jerusalem (IL)

(72) Inventors: Erez Shapir, Tzur Hadassa (IL); Hanna Friedman, Givat Zeev (IL)

(21) Appl. No.: 14/749,635

(22) Filed: Jun. 25, 2015

(51) Int. Cl. A61B 5/083; H01S 5/34

(52) U.S. Cl. CPC A61B 5/0836; H01S 5/3402

(57) ABSTRACT

The present disclosure provides a detector for a capnography device for uninterrupted monitoring of respiratory gasses of a subject, the detector includes a gas flow chamber, configured to permit flow of respiratory gasses between a first orifice and a second orifice thereof, a bandstructure cascade laser configured to provide mid-infrared wavelength laser radiation to the flow chamber, the mid-infrared wavelength laser radiation having wavelengths that at least partially correspond to an absorption spectrum of carbon-dioxide molecules, and a first radiation sensor configured to obtain mid-infrared wavelength radiation passing through the chamber and to provide a radiation intensity signal indicative of changes in carbon-dioxide level in respiratory gasses within the chamber during at least one respiration cycle.

### TECHNICAL FIELD

The present disclosure generally relates to the field of respiratory monitoring.

### BACKGROUND

Capnography is a non-invasive monitoring method used to continuously measure CO₂ levels (concentration/partial pressure) in respiratory gases. The CO₂, which is a constant metabolism product of the cells, is exhaled out of the body and the concentration of the exhaled CO₂, also known as end tidal CO₂ (EtCO₂) is an approximate estimation of the arterial levels of CO₂. The measurements of the CO₂ levels in a breath cycle are performed by a capnograph, and the results are values which may also be displayed in a graphical format in the shape of a waveform named a capnogram. The values, typically numerical value of the results, may be presented in units of pressure (for example, mm Hg) or percentile. The capnogram may depict CO₂ concentration against total expired volume, but the more common capnogram illustrates CO₂ concentration against time.

Measuring carbon-dioxide (CO₂) levels in respiratory gasses (capnography) is commonly done by radiating electromagnetic waves through a CO₂ containing gas and spectroscopically analyzing the electromagnetic waves after passing through the gas. CO₂ molecules have a known absorption spectrum, in which certain wavelengths are absorbed by the CO₂ molecules and others are not. By measuring the intensities of the electromagnetic waves at corresponding wavelengths after passing through the CO₂ containing gas and comparing it with a reference measurement of a non CO₂ containing gas, one can derive the concentration of CO₂ in the CO₂ containing gas.

Current capnography techniques use a gas discharge-tube or blackbody (BB) radiation sources, which provide a wide array of electromagnetic wavelengths, thus requiring a complex design, including infrared filters, chopper wheels and highly sophisticated data processing units to limit the wavelengths to a desired range, and correlate the CO₂ level to the intensity of light passing through the gas. The use of gas discharge-tubes, or blackbody radiation sources, results in a complex structure that requires a sophisticated spectral sensor to accurately measure the specific desired wavelengths.

There is thus a need in the art for capnography systems with a simple and accurate radiation source that provides electromagnetic radiation precisely at desired wavelengths.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided herein devices, systems and methods for capnographic monitoring using a detector having a bandstructure cascade laser (BCL) as a source of radiation. According to some embodiment, the term “bandstructure cascade laser (BCL)” may include a quantum cascade laser (QCL), interband cascade laser (ICL) and the like. The BCL is configured to provide stable electromagnetic radiation with high wavelength accuracy, without emitting electromagnetic radiation having undesired wavelengths or time variability. Advantageously, utilizing a BCL configured to provide accurate predetermined wavelength radiation may obviate the need to use a radiation filter and/or other signal processing components.

The energy consumed by the BCL is directed to generating radiation at specific wavelengths, contrary to gas discharge-tubes that generate a relatively wide spectrum of wavelengths. Advantageously, providing only desired wavelengths may reduce the power consumption of the detector in comparison to the use of conventional gas discharge-tube detectors.

According to some embodiments, utilizing a BCL as a radiation source enables providing radiation having two wavelengths, each corresponds to a specific CO₂ absorption wavelength. Advantageously, having two distinct wavelengths of radiation enables measuring CO₂ levels using two independent indications, and as a result may provide a more accurate and reliable measurement.

According to some embodiments, the BCL provides radiation at accurate wavelengths. Advantageously, having radiation at accurate wavelengths mitigates the variance of the measurements and may result in an improved capability of the device to maintain a strong absorption signal over variations in the CO₂ gas absorption band structure due to external conditions.

According to some embodiments, there is provided a carbon dioxide (CO₂) detector for a capnography device for uninterrupted monitoring of respiratory gasses of a subject, the detector including a gas flow chamber, configured to permit flow of respiratory gasses between a first orifice and a second orifice thereof, a bandstructure cascade laser configured to provide mid-infrared wavelength laser radiation to the flow chamber, the mid-infrared wavelength laser radiation having wavelengths that at least partially correspond to an absorption spectrum of carbon-dioxide molecules, and a first radiation sensor configured to obtain mid-infrared wavelength radiation passing through the chamber and to provide a radiation intensity signal indicative of changes in carbon-dioxide level in respiratory gasses within the chamber during at least one respiration cycle.

According to some embodiments, the flow of respiratory gasses is continuous.

According to some embodiments, the radiation is continuous.

According to some embodiments, the radiation is provided intermittently.

According to some embodiments, the radiation is provided at a rate of at least 1 MHz.

According to some embodiments, there is provided a capnography device for monitoring carbon dioxide (CO₂) in respiratory gasses of a subject, the device including a gas flow chamber, configured to permit flow of respiratory gasses between a first orifice and a second orifice thereof, a bandstructure cascade laser configured to provide mid-infrared wavelength laser radiation to the flow chamber, the mid-infrared wavelength laser radiation having wavelengths that at least partially correspond to an absorption spectrum of carbon-dioxide molecules, a first radiation sensor configured to obtain mid-infrared wavelength radiation passing through the chamber and to provide a radiation intensity signal indicative of changes in carbon-dioxide level in respiratory gasses within the chamber during at least one respiration cycle, and a processing circuitry configured to analyze the radiation intensity signal and to derive a carbon-dioxide waveform corresponding to at least one respiration cycle of the subject.

According to some embodiments, the mid-infrared wavelength laser is a laser having wavelengths from 3 microns to 15 microns.

According to some embodiments, the bandstructure cascade laser is configured to provide radiation having a wavelength of 4.2 microns.

According to some embodiments, the bandstructure cascade laser is configured to provide radiation characterized by two wavelengths corresponding to two absorption wavelengths in the absorption spectrum of carbon-dioxide.

According to some embodiments, the sensor is configured to provide at least two signals, each independently indicative of changes in carbon-dioxide levels in respiratory gasses within the chamber by distinctly sensing radiation intensities of the two wavelengths.

According to some embodiments, the device further includes a second sensor configured to provide a radiation intensity signal relating to a second wavelength of the two wavelengths, and the first sensor is configured to provide a radiation intensity signal relating to a first wavelength of the two wavelengths.

According to some embodiments, the device further includes a processing circuitry configured to analyze the radiation intensity signal and to derive a waveform of carbon-dioxide level corresponding to at least one respiration cycle.

According to some embodiments, the radiation intensity signal is continuous and provides continuous indication of carbon-dioxide level in respiratory gasses within the chamber during at least one respiration cycle.

According to some embodiments, the radiation intensity signal is a discrete signal including multiple samples indicative of carbon-dioxide levels at various times in respiratory gasses within the chamber during at least one respiration cycle.

According to some embodiments, the multiple samples have a sampling rate of more than 50 samples per minute, for example, more than 100 samples per minute, more than 150 samples per minute or more than 200 samples per minute. According to some embodiments, multiple samples have a sampling rate of more than 100 samples per respiration cycle.

According to some embodiments, the bandstructure cascade laser is configured to provide pulses of radiation in times and rate corresponding to sampling times and a sampling rate of the sensor.

According to some embodiments, the device further includes a collimator, configured to narrow a scattering of the radiation provided by the bandstructure cascade laser before reaching the chamber.

According to some embodiments, the device further includes a monitor configured to display a carbon-dioxide waveform derived by the processing circuitry.

According to some embodiments, there is provided a method for monitoring carbon dioxide in respiratory gasses of a subject, the method including continuously flowing respiratory gasses between a first orifice and a second orifice of a flow chamber, irradiating the flow chamber with mid-infrared wavelength laser radiation produced by a bandstructure cascade laser such that the mid-infrared wavelength laser radiation is at least partially absorbed by carbon-dioxide present in the respiratory gasses, and using a first radiation sensor, detecting an intensity of mid-wavelength infrared radiation passing through the flow chamber and providing a radiation intensity signal indicative of changes in carbon-dioxide level in respiratory gasses within the flow chamber.

According to some embodiments, the method further includes analyzing, using a processing circuitry, the radiation intensity signal and deriving a carbon-dioxide waveform corresponding to at least one respiration cycle of the subject.

According to some embodiments, the bandstructure cascade laser is configured to irradiate the flow chamber continuously.

According to some embodiments, the bandstructure cascade laser is configured to irradiate the flow chamber intermittently.

According to some embodiments, the bandstructure cascade laser comprises a quantum cascade laser and/or an interband cascade laser.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a conventional capnographic system with a gas discharge-tube as a light source;

FIG. 2 schematically illustrates a capnographic system with a BCL as a detector light source, according to some embodiments;

FIG. 3a schematically illustrates a BCL radiation scheme, according to some embodiments;

FIG. 3b schematically illustrates a BCL radiation scheme with a collimator, according to some embodiments;

FIG. 4a schematically illustrates a radiation spectrum of a BCL, according to some embodiments;

FIG. 4b schematically illustrates a radiation spectrum of a BCL radiating at two wavelengths, according to some embodiments; and

FIG. 5 schematically illustrates intermittent/fragmented mid-infrared laser radiation and sensing of radiation intensities at a sensor, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

CO₂ concentration detection is commonly done by radiating electromagnetic waves to a CO₂ containing gas and analyzing the absorption of the radiation by measuring the spectral intensity of the radiation by a spectral sensor.

According to some available techniques, the radiation source is a gas discharge-tube or black body source that emits radiation in a wide range of wavelengths, and a sophisticated spectral sensor is used to measure the intensities of radiation across a wide range of wavelengths and isolate a relevant wavelength that correlates to the CO₂ absorption spectrum. These sophisticated spectral sensors require optical filters and complex data processing, which are expensive and sometimes may not be sufficiently accurate for capnography.

According to other available techniques, the radiation source is a gas discharge-tube that emits radiation in a wide range of wavelengths; and wavelength filters are utilized to allow transmission radiation with desired wavelengths to obstruct the transmission of radiation with other wavelengths. These techniques suffer from dependence on radiation filters and complex data processing and possibly cause a waste of energy by producing radiation having wavelengths that are not used in the absorption/measuring process.

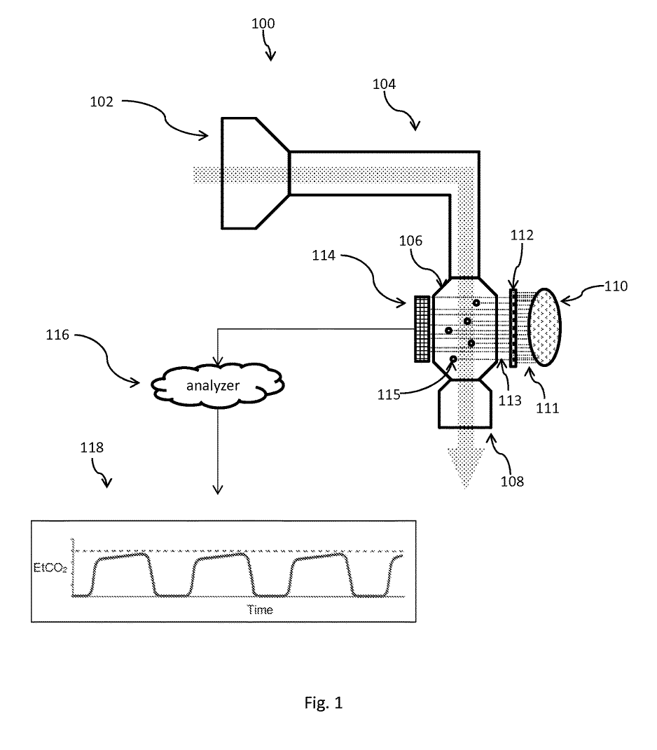

Reference is now made to FIG. 1, which schematically illustrates a conventional capnography system 100 with a gas discharge-tube 110. Capnography system 100 has a user interface 102 for obtaining respiratory gasses from a subject. User interface 102 is connected to a tubing system 104 that delivers samples of the respiratory gasses from a subject to a measurement chamber 106, and from there the gasses pass to an air-passage 108. Gas discharge-tube 110 radiates electromagnetic waves 111 that pass through a filter 112 configured to pass electromagnetic waves having wavelengths that fall in a predetermined range of wavelengths, resulting in filtered electromagnetic waves 113. Filtered electromagnetic waves 113 pass through measurement chamber 106, and some of the waves are absorbed by carbon dioxide molecules 115 present in the respiratory gasses at measurement chamber 106.

At least some waves of filtered electromagnetic waves 113 do not get absorbed/obstructed by carbon dioxide molecules 115 and reaches sensor 114 that is configured to sense the intensity of obtained radiation and send a signal to an analyzer 116 that analyzes the intensities and displays the results on a display 118.

According to other available techniques, electromagnetic waves are radiated from an IR source based on blackbody radiation behavior, which results in radiating a relatively wide spectrum of wavelengths, which requires heating a radiation source to high temperatures, typically around 300-800° C. and also necessitates utilizing radiation filters and chopper wheels, such that only electromagnetic waves having desired wavelengths reach the measurement chamber, and to provide reference signal.

Advantageously, according to some embodiments, utilizing a bandstructure cascade laser (BCL) to radiate electromagnetic waves does not require heating to high temperature, therefore, the consumed energy by the BCL may be lower compared to the energy consumed by a Blackbody IR source.

As used herein, According to some embodiments, the term “bandstructure cascade laser” (BCL) refers to a cascade laser in which energy levels in a quantum wells structure are engineered by a process known as bandstructure engineering to create a desired energy differentiation between different quantum energy levels in active regions. When an electron is injected to the structure, the energy differentiation results in an emission of a photon having an energy, determined by the energy differentiation between the different quantum energy levels. By cascading multiple active regions, a single electron may pass through the cascade, thereby emitting multiple photons at desired energies (wavelengths).

According to some embodiments, the energy differentiation between quantum energy levels in the active region is in the range of 1.4 meV to 1.7 eV. According to come embodiments, the energy differentiation between quantum energy levels in the active region is in the range of 155 meV to 886 meV resulting in the emission of photons having a wavelength range of 8 μm (micro-meter) to 1.4 μm (micro-meter)). According to come embodiments, the energy differentiation between quantum energy levels in the active region is in the range of 290 meV to 300 meV resulting in the emission of photons having a wavelength range of 4.27 μm (micro-meter) to 4.13 μm (micro-meter)). According to come embodiments, the energy differentiation between quantum energy levels in the active region is approximately 290 meV resulting in the emission of photons having a wavelength of approximately 4.2 μm (micro-meter)).

According to some embodiments, the BCL may include an interband transition mechanism for emitting photons; such lasers may include an interband cascade laser (ICL).

According to some embodiments, the BCL may include an intersubband transition mechanism for emitting photons; such lasers may include a quantum cascade laser (QCL).

According to some embodiments, the BCL is configured to provide stable electromagnetic radiation with high wavelength accuracy, without emitting electromagnetic radiation having undesired wavelengths or time variability. Advantageously, utilizing a BCL configured to provide accurate predetermined wavelength radiation may obviate the need to use a radiation filter and/or other signal processing components.

According to some embodiments, the off-to-on time (rise time) of the BCL is less than 1 ms. According to some embodiments, the BCL is configured to be characterized with a rise time of up to 1 micro second. According to some embodiments, the BCL is configured to be characterized with a rise time in the range of 0.1 ns to 1000 ns. According to some embodiments, the BCL is configured to be characterized with a rise time in the range of 1 ns to 100 ns. According to some embodiments, the BCL is configured to be characterized with a rise time in the range of 2 ns to 50 ns. According to some embodiments, the BCL is configured to be characterized with a rise time in the range of 5 ns to 10 ns. According to some embodiments, the BCL is configured to be characterized with a rise time of less than 1 ns.

Advantageously, the fast rise-time of the BCL may facilitate fine modulation of the radiated laser.

Advantageously, the fast rise-time of the BCL may enable intermittent sampling, thereby result in a lower energy consumption.

According to some embodiments, the BCL is configured to have a quantum efficiency greater than a unity, thereby providing a high power-efficiency lasing. Advantageously, such a BCL laser may operate while consuming less energy compared with current capnography radiation sources, such as discharge-tubes.

According to some embodiments, the BCL is configured to operate at a power consumption of approximately 100 μW (micro-Watt). According to some embodiments, the BCL is configured to operate at a power consumption of approximately 10 μW (micro-Watt). According to some embodiments, the BCL is configured to operate at a power consumption in the range of 1 μW (micro-Watt) to 1 W (Watt). According to some embodiments, the BCL is configured to operate at a power consumption in the range of 1 μW (micro-Watt) to 100 μW (micro-Watt). According to some embodiments, the BCL is configured to operate at a power consumption in the range of 10 μW (micro-Watt) to 50 μW (micro-Watt). According to some embodiments, the BCL is configured to operate at a power consumption in the range of 100 μW (micro-Watt) to 900 μW (micro-Watt).

Advantageously, the BCL is configured to operate and facilitate capnography at a higher power-efficiency compared with current capnography radiation sources, such as a discharge-tube.

According to some embodiments, the BCL is configured to be a tunable BCL. According to some embodiments, a tunable BCL is a BCL in which the wavelength(s) of the produced laser(s) is(are) controllable/tunable.

According to some embodiments, the BCL is a distributed feedback laser. According to some embodiments the distributed feedback laser includes a distributed Bragg reflector (DBR) configured to facilitate tunability of the wavelengths of the radiated laser.

According to some embodiments, the BCL is an external cavity (EC) BCL, for example an EC-QCL. According to some embodiments, the EC-BCL utilizes diffraction grading to facilitate tenability of the wavelengths of the radiated laser.

According to some embodiments, the BCL includes a temperature control mechanism, and the control over the wavelengths of the BCL is done by changing/manipulating the temperature of the BCL using the temperature control unit.

According to some embodiments, the BCL includes a voltage control mechanism, and the control over the wavelengths of the BCL is done by changing/manipulating the voltage over the BCL, using the voltage control unit.

Advantageously, the tune-ability of the BCL is availed for increasing the accuracy of the capnography, for example, by tuning the wavelengths to target the highest absorption rate of carbon-dioxide, which may vary depending on environmental conditions such as pressure, temperature, externally induced fields and others.

According to some embodiments, the tunable BCL is configured to radiate at a plurality of wavelengths at different periods of times, such that at a certain period of time the tunable BCL is tuned to radiate at a certain wavelength, while at another period of time it is tuned to radiate at a different wavelength. According to some embodiments, the plurality of wavelengths is within a range close to a peak absorption wavelength of the carbon-dioxide. According to some embodiments, the plurality of wavelengths is in the range of 4.0 μm (micro-meter) to 4.4 μm (micro-meter). According to some embodiments, the plurality of wavelengths is in the range of 4.1 μm (micro-meter) to 4.3 μm (micro-meter).

According to some embodiments, the tunable BCL is tuned to radiate at a first wavelength at some time periods, and be tuned to radiate at a second wavelength at other time periods. According to some embodiments, the first wavelength is approximately 4.21 μm (micro-meter) and the second wavelength is approximately 4.19 μm (micro-meter). According to some embodiments, the first wavelength is approximately 4.22 μm (micro-meter) and the second wavelength is approximately 4.18 μm (micro-meter). According to some embodiments, the first wavelength is approximately 2.71 μm (micro-meter) and the second wavelength is approximately 2.69 μm (micro-meter). According to some embodiments, the first wavelength is approximately 2.72 μm (micro-meter) and the second wavelength is approximately 2.68 μm (micro-meter).

According to some embodiments, the tunable BCL is tuned to radiate at a first wavelength at some time periods, tuned to radiate at a second wavelength at other time periods and tuned to radiate at a third wavelength at different, yet other, time periods. According to some embodiments, the first wavelength is approximately 4.21 μm (micro-meter), the second wavelength is approximately 4.19 μm (micro-meter) and the third wavelength is approximately 4.2 μm (micro-meter). According to some embodiments, the first wavelength is approximately 4.22 μm (micro-meter), the second wavelength is approximately 4.18 μm (micro-meter) and the third wavelength is approximately 4.2 μm (micro-meter).

Advantageously, radiating at different wavelengths at different time periods may increase the resolution of the detection. Advantageously, radiating at different wavelengths at different time periods may increase the accuracy of the detection.

According to some embodiments of the disclosure, a bandstructure cascade laser (BCL) is utilized to radiate electromagnetic waves at desired wavelengths. Advantageously, the BCL provides electromagnetic radiation with a low variance in the wavelengths thereof, which may obviate the need to use a filter such as filter 112 as illustrated in FIG. 1.

Advantageously, using a BCL may bring fast turning on and turning off times compared to a gas discharge-tube.

The currently available capnography radiation sources are prone to drifts and variations in their radiated wavelengths over time, which imposes a technical challenge on the detection and analysis of the detected signal. Additionally, the absorption spectrum of the CO₂ molecules varies depending on multiple conditions, such as temperature, pressure, flow and others. Current analysis units need to take into account both the variance in the radiated wavelengths and the variance in the absorption spectrum of the CO₂ molecules, which requires complex algorithms, in addition to suffering from a reduced accuracy of measurements.

Advantageously, utilizing a BCL as a radiation source for capnography reduces the variations and drifts of the radiated wavelengths, thereby allowing the use of a less complex analysis unit and less complex algorithms, in addition to obtaining higher measurement accuracy.

According to some embodiments, the BCL is configured to provide coherent radiation. Advantageously, the coherent radiation may enable utilizing a smaller chamber and, as a result, achieve a higher sampling rate compared with current capnography devices, without compromising the signal to noise ratio of the detection.

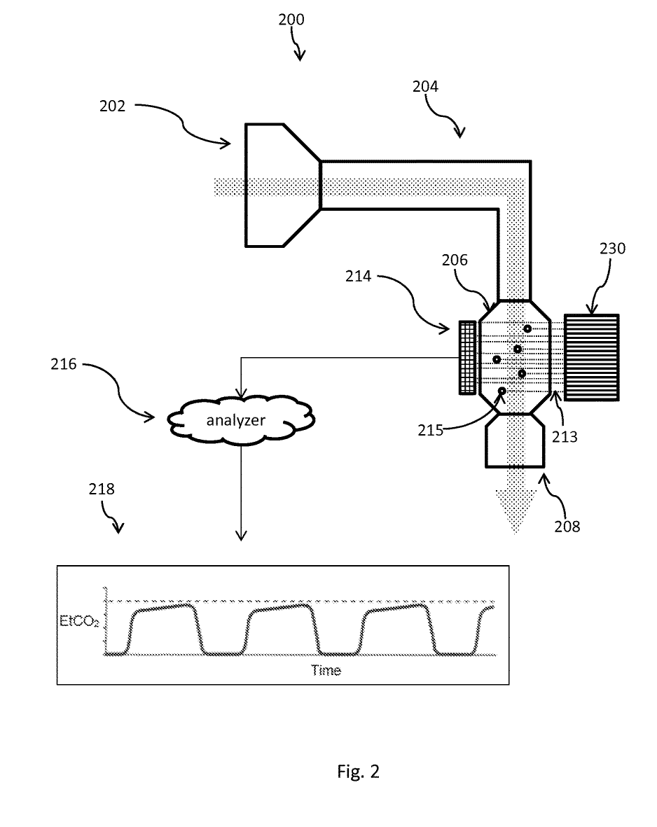

Reference is now made to FIG. 2, which schematically illustrates a capnography system 200 with a BCL 230, according to some embodiments. Capnography system 200 has a user interface 202 for obtaining samples of respiratory gasses from a subject. User interface 202 is connected to a tubing system 204 that delivers the samples of respiratory gasses from a subject to a measurement chamber 206 and from there the gasses reach an air-passage 208. Bandstructure Cascade Laser (BCL) 230 radiates electromagnetic waves 213 having predetermined wavelengths. Electromagnetic waves 213 pass through measurement chamber 206, and some of the waves are absorbed by carbon dioxide molecules 215.

At least some of electromagnetic waves 213 do not get absorbed by carbon dioxide molecules 215 and reaches sensor 214 that is configured to sense the intensity of obtained radiation and send a signal to an analyzer 216 that analyzes the intensities and displays the results on a display 218.

If measurement chamber 206 has high concentrations of carbon dioxide molecules 215, a large portion of electromagnetic waves 213 is absorbed and only a small portion reaches sensor 214. Sensor 214 send a weak intensity signal to analyzer 216, indicating that the concentration of carbon dioxide molecules 215 in chamber 206 is high. Alternatively, if chamber 206 contains low concentrations of carbon dioxide molecules 215, a small portion of electromagnetic waves 213 is absorbed and a large portion reaches sensor 214. Sensor 214 sends a strong intensity signal to analyzer 216, indicating that the concentration of carbon dioxide molecules 215 in chamber 206 is low.

According to some embodiments, tubing system 204 is configured to deliver samples of respiratory gas to measurement chamber 206. According to some embodiments, tubing system 204 includes a cannula.

According to some embodiments capnography system 200 is configured to allow continuous breathing of the subject, and BCL 230 is configured to provide a continuous uninterrupted radiation of electromagnetic waves 213 at desired wavelengths, and sensor 214 is configured to continuously provide intensity measurements to analyzer 216 to track changes in carbon dioxide concentration changes during respiration cycles of the subject.

According to some embodiments, BCL 230 is configured to provide an interrupted/fragmented radiation of electromagnetic waves 213 at desired wavelengths in a predetermined and/or configurable radiation rate, and sensor 214 is configured to provide sampled (discrete) intensity measurements to analyzer 216 at a predetermined and/or configurable sampling rate, synchronized with the radiation rate of BCL 230. According to some embodiments, analyzer 216 obtains the sampled (discrete) signal from sensor 214 and extrapolates a continuous carbon dioxide concentration chart, indicating changes of the concentration during respiration cycles of the subject.

According to some embodiments, the sampling rate and/or the radiation rate is approximately 20 Hz. According to some embodiments, the sampling rate and/or the radiation rate is less than 100 Hz and more than 10 Hz. According to some embodiments, the sampling rate and/or the radiation rate is less than 50 Hz and more than 15 Hz.

According to some embodiments, the desired wavelengths correspond to the absorption spectrum of carbon dioxide molecules. According to some embodiments, desired wavelengths are approximately 4.2 μm (micro-meter). According to some embodiments, desired wavelengths are approximately 2.7 μm (micro-meter). According to some embodiments, desired wavelengths are approximately 2.7 μm (micro-meter) and 4.2 μm (micro-meter) simultaneously.

According to some embodiments, the BCL is configured to radiate electromagnetic waves at approximately 4.2 μm (micro-meter) with variance of approximately 1.0 nm to 2.0 nm. According to some embodiments, the BCL is configured to radiate electromagnetic waves at approximately 4.2 μm (micro-meter) with variance of less than 10 nm.

According to some embodiments, measurement chamber 206 is configured to non-obstructively allow flow of respiratory gasses therethrough. According to some embodiments, measurement chamber 206 is made from materials that do not absorb mid-infrared radiation. According to some embodiments, measurement chamber 206 is made from quartz.

According to some embodiments, measurement chamber 206 has a cross section area of approximately 10 mm².

According to some embodiments, sensor 214 is a Thermopile, a pyroelectric infrared sensor or an HgCdTe infrared photodiode. Each possibility is a separate embodiment of the invention.

According to some embodiments, BCL 230 is configured to provide electromagnetic radiation at intensity of approximately 5 mW. According to some embodiments, BCL 230 is configured to provide electromagnetic radiation at intensity of approximately 50 mW. According to some embodiments, BCL 230 is configured to provide electromagnetic radiation at intensity of approximately 500 mW.

According to some embodiments, BCL 230 has a peak power consumption of 0.5 W to 10 W. According to some embodiments, BCL 230 has a peak power consumption of approximately 5 W.

According to some embodiments, BCL 230 has a turn-on time of approximately 10 ns. According to some embodiments, BCL 230 has a turn-on time of approximately 100 ns.

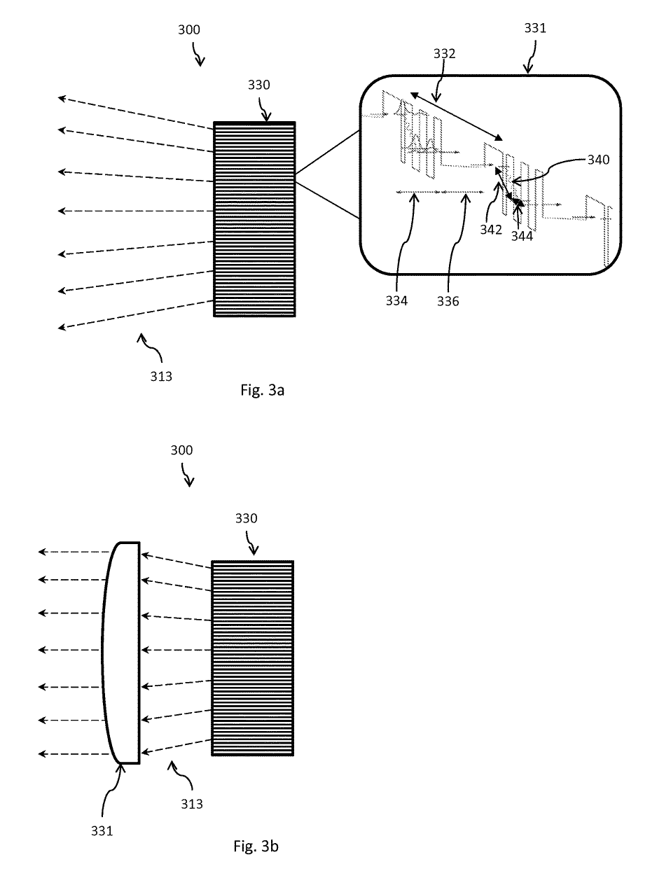

Reference is now made to FIG. 3a, which schematically illustrates a radiation scheme 300 with a BCL 330, according to some embodiments. BCL 330 comprises multiple cascaded segments. A schematic zoom view 331 of BCL 330 illustrates a segment 332 having an active region 334 configured to emit photons 340 at a predetermined wavelength depending on an energy gap 342, and a phonon is released from a following energy gap 344. Segment 332 further comprises a regeneration region 336 configured to enable a buildup of energy before reaching an active region of a neighboring segment.

The photons emitted from the cascade of all segments are radiated and provide electromagnetic waves 313 having predetermined wavelengths.

Reference is now made to FIG. 3b, which schematically illustrates a radiation scheme 300 of a BCL 330 with a collimator 331, according to some embodiments. Electromagnetic waves produced by BCL 330 are generally scattered, and may diffuse and reach a corresponding sensor with low intensities. Collimator 331 is configured to narrow and concentrate the radiation provided by BCL 330 in the direction of corresponding sensor(s).

Generally, the absorption spectrum of CO₂ shows high absorptivity for electromagnetic waves having wavelengths of approximately: 2.7 μm (micro-meter), 4.2 μm (micro-meter) and 15 μm (micro-meter). This implies that providing electromagnetic waves at these wavelengths may enable detection of CO₂ molecule concentration in a gas mixture, through which the electromagnetic waves pass. If the intensity of electromagnetic waves that successively pass through the gas is high, it indicates that the concentration of CO₂ molecules in the gas is low. Alternatively, if the intensity of electromagnetic waves that successively pass through the gas is low, it indicates that the concentration of CO₂ molecules in the gas mixture is high.

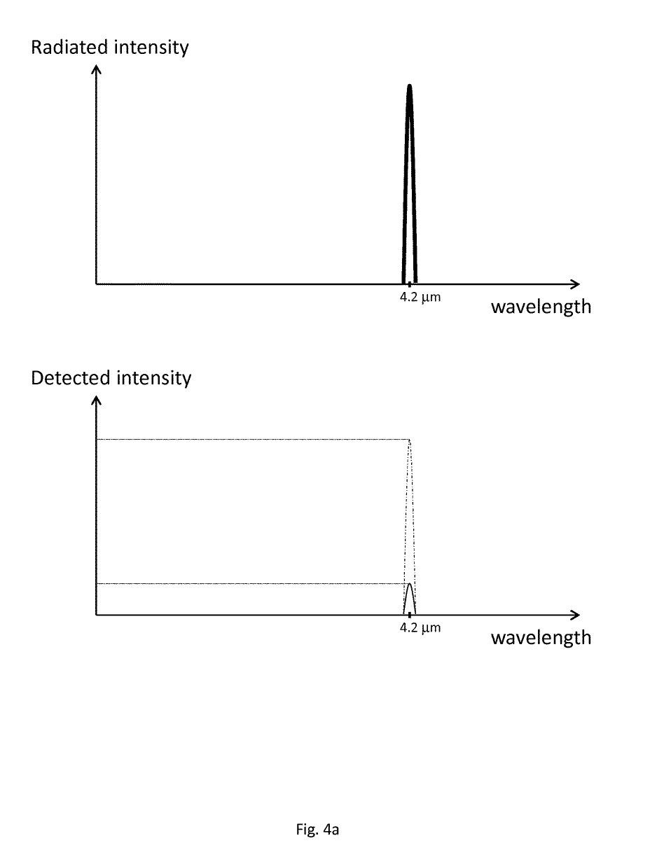

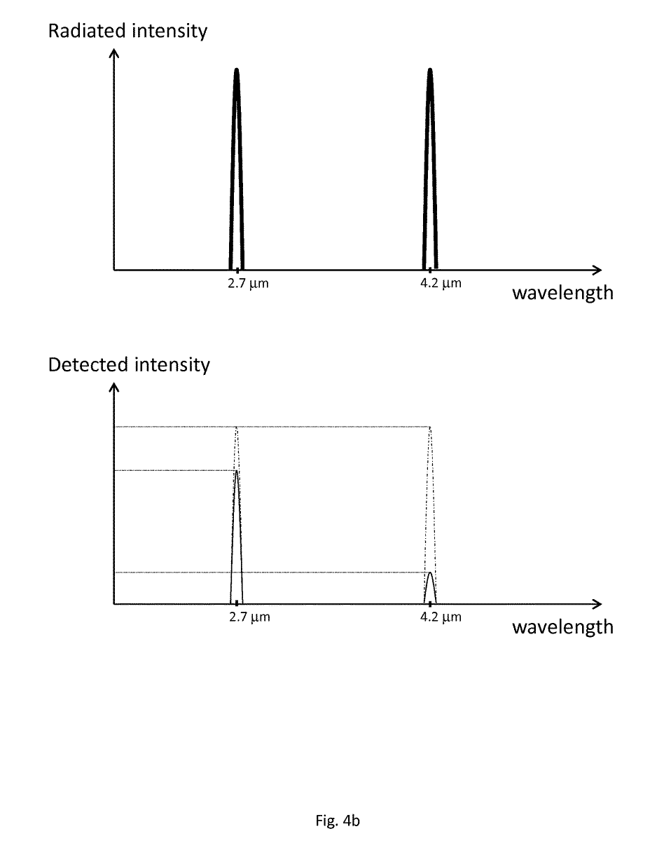

Reference is now made to FIG. 4a, which schematically illustrates a radiation spectrum of a BCL, according to some embodiments. The upper graph illustrates an exemplary radiated intensity of a BCL configured to provide electromagnetic waves at wavelength of 4.2 μm (micro-meter), and the lower graph illustrates exemplary detected intensities at a sensor associated with the BCL. The low detected intensity indicates that the concentration of CO₂ molecules in the gas is high as a large portion of the waves was absorbed, and the high detected intensity indicates that the concentration of CO₂ molecules in the gas is low as only a small portion of the waves was absorbed.

Reference is now made to FIG. 4b, which schematically illustrates a radiation spectrum of a BCL, according to some embodiments. The upper graph illustrates an exemplary radiated intensity of a BCL configured to provide electromagnetic waves at wavelengths of 4.2 μm (micro-meter) and 2.7 μm (micro-meter) at similar intensities for exemplary purposes, and the lower graph illustrates exemplary detected intensities at a sensor/s associated with the BCL. While high and low detected intensities are indicative of the concentration of CO₂ molecules in the gas (as in FIG. 4a), the sensor/s provides two separate detected intensity signals, one for a 4.2 μm (micro-meter) wavelength and one for a 2.7 μm (micro-meter) wavelength. It is noted that the detected signal at 2.7 μm (micro-meter) wavelength may be different from the detected signal at wavelength of 4.2 μm (micro-meter) as the absorption of radiation in carbon dioxide differs between the two wavelengths. As for the exemplary radiation of 4.2 μm (micro-meter) and 2.7 μm (micro-meter) at similar intensities, the 4.2 μm (micro-meter) radiation may be absorbed more than the 2.7 μm (micro-meter) radiation, therefore the detected intensity of 2.7 μm (micro-meter) wavelength radiation may be higher than the detected intensity of 4.2 μm (micro-meter) wavelength radiation.

According to some embodiments, multiple BCL structures may be utilized. According to some embodiments, there are provided two BCL structures, each configured to provide electromagnetic waves at different wavelengths. According to some embodiments, the first BCL structure is configured to provide electromagnetic waves at a wavelength of approximately 4.2 μm (micro-meter), while the second BCL structure is configured to provide electromagnetic waves at a wavelength of approximately 2.7 μm (micro-meter).

Advantageously, providing radiation at two distinct wavelengths may result in a lower drift and therefore achieve better accuracy and robustness of the capnography measurements.

According to some embodiments, there are provided three BCL structures, each configured to provide electromagnetic waves at different wavelengths. According to some embodiments, the first BCL structure is configured to provide electromagnetic waves at a wavelength of approximately 4.2 μm (micro-meter), the second BCL structure is configured to provide electromagnetic waves at a wavelength of approximately 2.7 μm (micro-meter) and the third BCL structure is configured to provide electromagnetic waves at a wavelength of approximately 15 μm (micro-meter).

Advantageously, providing radiation in at least two distinct wavelengths may result in a higher sensitivity of the capnography measurements.

According to some embodiments, the BCL may radiate pulses of electromagnetic waves, and the sensor associated thereto measures discrete intensity signals. An analyzing unit (analyzer) may then obtain the discrete intensity signal and extrapolate/derive a continuous capnogram (CO₂ molecules concentration) graph therefrom.

According to some embodiments, the BCL is configured to radiate pulses at a pulse rate of approximately 10 MHz. According to some embodiments, the BCL is configured to radiate pulses at a pulse rate of more than 1 MHz. According to some embodiments, the BCL is configured to radiate pulses at a pulse rate of 1 MHz to 50 MHz. According to some embodiments, the BCL is configured to radiate pulses at a pulse rate of 10 MHz to 100 MHz. According to some embodiments, the BCL is configured to radiate pulses at a pulse rate of 50 MHz to 200 MHz.

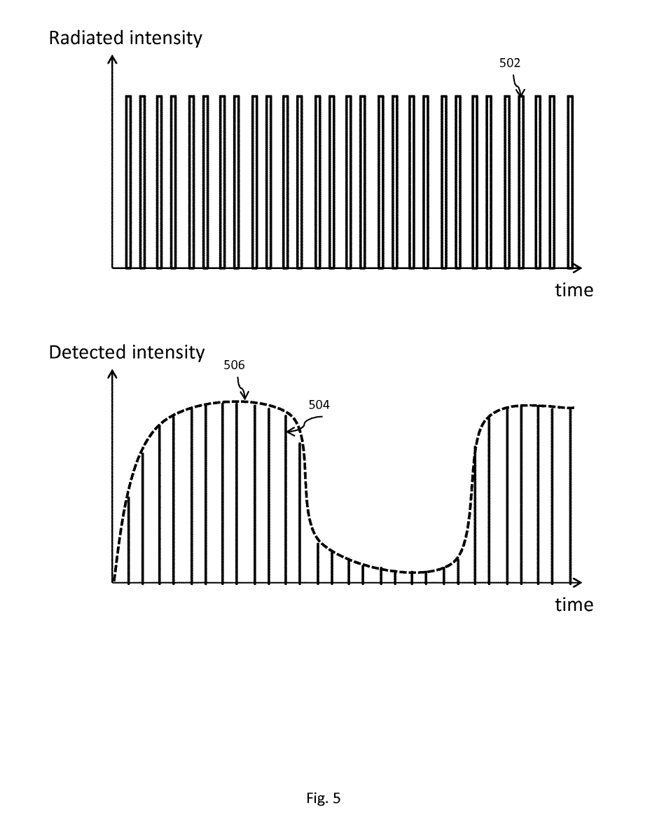

Reference is now made to FIG. 5, which schematically illustrates intermittent/fragmented radiation and sensing, according to some embodiments. The upper graph illustrates an exemplary radiated intensity of a BCL configured to provide electromagnetic wave pulses resulting in fragmented radiation 502, and the lower graph illustrates exemplary discrete intensities 504 detected by sensor/s associated with the BCL and a continuous capnogram (CO₂ molecules concentration) 506 extrapolated from discrete intensities 504.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A carbon dioxide (CO₂) detector for a capnography device for uninterrupted monitoring of respiratory gasses of a subject, the detector comprising:
a gas flow chamber, configured to permit flow of respiratory gasses between a first orifice and a second orifice thereof;
a bandstructure cascade laser configured to provide mid-infrared wavelength laser radiation to said flow chamber, the mid-infrared wavelength laser radiation having wavelengths that at least partially correspond to an absorption spectrum of carbon-dioxide molecules; and
a first radiation sensor configured to obtain mid-infrared wavelength radiation passing through said chamber and to provide a radiation intensity signal indicative of changes in carbon-dioxide level in respiratory gasses within said chamber during at least one respiration cycle.

2. The detector of claim 1, wherein the flow of respiratory gasses is continuous.

3. The detector of claim 1, wherein the radiation is continuous.

4. The detector of claim 1, wherein the radiation is provided intermittently.

5. The detector of claim 4, wherein the radiation is provided at a rate of at least 1 MHz.

6. The detector of claim 1, wherein the bandstructure cascade laser comprises a quantum cascade laser and/or an interband cascade laser.

7. A capnography device for monitoring carbon dioxide (CO₂) in respiratory gasses of a subject, the device comprising:
a gas flow chamber, configured to permit flow of respiratory gasses between a first orifice and a second orifice thereof;
a bandstructure cascade laser configured to provide mid-infrared wavelength laser radiation to said flow chamber, the mid-infrared wavelength laser radiation having wavelengths that at least partially correspond to an absorption spectrum of carbon-dioxide molecules;
a first radiation sensor configured to obtain mid-infrared wavelength radiation passing through said chamber and to provide a radiation intensity signal indicative of changes in carbon-dioxide level in respiratory gasses within said chamber during at least one respiration cycle; and
a processing circuitry configured to analyze said radiation intensity signal and to derive a carbon-dioxide waveform corresponding to at least one respiration cycle of the subject.

8. The device of claim 7, wherein the mid-infrared wavelength laser is a laser having wavelengths from 3 microns to 15 microns.

9. The device of claim 7, wherein said bandstructure cascade laser is configured to provide radiation having a wavelength of 4.2 microns.

10. The device of claim 7, wherein said bandstructure cascade laser is configured to provide radiation characterized by two wavelengths corresponding to two absorption wavelengths in the absorption spectrum of carbon-dioxide.

11. The device of claim 10, further comprising a second sensor configured to provide a radiation intensity signal relating to a second wavelength of the two wavelengths and said first sensor is configured to provide a radiation intensity signal relating to a first wavelength of the two wavelengths.

12. The device of claim 7, further comprising a processing circuitry configured to analyze said radiation intensity signal and to derive a waveform of carbon-dioxide level corresponding to at least one respiration cycle.

13. The device of claim 7, wherein the radiation intensity signal is continuous and provides continuous indication of carbon-dioxide level in respiratory gasses within said chamber during at least one respiration cycle.

14. The device of claim 7, wherein the radiation intensity signal is a discrete signal comprising multiple samples indicative of carbon-dioxide levels at various times in respiratory gasses within said chamber during at least one respiration cycle.

15. The device of claim 14, wherein the multiple samples have a sampling rate of more than 50 samples per minute.

16. The device of claim 7, further comprising a collimator, configured to narrow a scattering of the radiation provided by said bandstructure cascade laser before reaching said chamber.

17. The device of claim 7, further comprising a monitor configured to display a carbon-dioxide waveform derived by said processing circuitry.

18. The device of claim 7, wherein the bandstructure cascade laser comprises a quantum cascade laser and/or an interband cascade laser.

19. A method for monitoring carbon dioxide in respiratory gasses of a subject, the method comprising:
continuously flowing respiratory gasses between a first orifice and a second orifice of a flow chamber;
irradiating the flow chamber with mid-infrared wavelength laser radiation produced by a bandstructure cascade laser such that the mid-infrared wavelength laser radiation is at least partially absorbed by carbon-dioxide present in the respiratory gasses; and
using a first radiation sensor, detecting an intensity of mid-wavelength infrared radiation passing through the flow chamber and providing a radiation intensity signal indicative of changes in carbon-dioxide level in respiratory gasses within the flow chamber.

20. The method of claim 19, further comprising analyzing, using a processing circuitry, the radiation intensity signal and deriving a carbon-dioxide waveform corresponding to at least one respiration cycle of the subject.
